(19) United States
(12) Patent Application Publication
(10) Pub. No.: US 2020/0193596 A1
(43) Pub. Date: Jun. 18, 2020

(54) Method And System For Identifying And Classifying Structures In A Blood Sample

(71) Applicant: Hemotech Cognition, LLC, Austin, TX (US)

(72) Inventor: Theodore F. BAYER, Austin, TX (US)

(73) Assignee: Hemotech Cognition, LLC, Austin, TX (US)

(21) Appl. No.: 16/719,360

(22) Filed: Dec. 18, 2019

Related U.S. Application Data
(60) Provisional application No. 62/781,168, filed on Dec. 18, 2018.

(51) Int. Cl. G06T 7/00; G01N 33/49; G06K 9/00

(52) U.S. Cl. CPC G06T 7/0012; G01N 33/49; G06K 9/00147; G06T 2207/30004; G06T 2207/20084; G06T 2207/10056; G06T 2207/10148; G06T 2207/10016

(57) ABSTRACT

A system and method for determining types of objects within a bodily fluid sample includes a sample holder holding a bodily fluid sample, an image capture device generating a plurality of images of the sample, and a sample positioner positioning the sample holder. An image capture device generates a plurality of images of the sample at a plurality of positions. A trained image classifier the plurality of images at a trained image classifier to identify a type of objects in the bodily fluid sample. An analyzer, that in response to classifying, displays an indicator on a display for indicating the type of objects is present within the bodily fluid sample.

### CROSS-REFERENCE TO RELATED APPLICATIONS

This application claims the benefit of U.S. Provisional Application No. 62/781,168, filed on Dec. 18, 2018. The entire disclosure of the above application is incorporated herein by reference.

### TECHNICAL FIELD

The present disclosure relates generally to blood content diagnosis systems and, more specifically, to a method and system providing identifying and classifying structures in a blood sample.

### BACKGROUND

The statements in this section merely provide background information related to the present disclosure and may not constitute prior art.

It is important for doctors to be able to determine what type of pathogen a person may have. Also, knowing the severity of the pathogen is also desirable. Various types of pathogens may be determined in different ways. The use of cultures, antibodies, antigens or genetic material are often used in various tests.

Different types of bodily fluids may be examined such as blood, sputum, urine, stool, tissue, cerebral spinal fluid and mucus from the nose, throat or genital area.

Conventional determination of pathogens within various bodily fluids may not be adequate for screening pathogens such as Lyme disease. Lyme disease is blood borne disease with spirochete or biofilm present in Lyme disease samples. The identification of Lyme disease is often difficult.

### SUMMARY

The present disclosure provides a way to analyze blood of other bodily fluids in an optical manner using a machine learning model to detect objects or structures. Optical recognition in a bodily fluid such as blood is performed by a trained machine learning model that recognizes the shapes, object or structures of pathogens.

In one aspect of the disclosure, system for classifying for determining types of objects within a bodily fluid sample includes a sample holder holding a bodily fluid sample, an image capture device generating a plurality of images of the sample, and a sample positioner positioning the sample holder. An image capture device generates a plurality of images of the sample at a plurality of positions. A trained image classifier the plurality of images at a trained image classifier to identify a type of objects in the bodily fluid sample. An analyzer, that in response to classifying, displays an indicator on a display for indicating the type of objects is present within the bodily fluid sample.

In another aspect of the disclosure, a method of identifying objects comprises controlling a position of an image capture device relative to a bodily fluid sample, generating a plurality of images from the bodily fluid sample at a plurality of different positions, classifying the plurality of images at a trained image classifier to identify a type of object in the bodily fluid sample and, in response to classifying, displaying an indicator on a display associated with an analyzer for indicating the type of object is present within the bodily fluid sample.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a block diagrammatic view of the system.

FIG. 2 is a flow chart of a method for detecting a pathogen.

FIG. 3 is a flow chart of a method for obtaining images.

FIG. 4 is a flow chart of a method for training a machine learning module.

FIG. 5 is a flow chart of a method for obtaining a sample.

FIG. 6 is a screen display for the analyzer.

FIG. 7A is a table of data used in training the classifier.

FIG. 7B is a confusion chart for the example of FIG. 7A

### DETAILED DESCRIPTION

The following description is merely exemplary in nature and is not intended to limit the present disclosure, application, or uses. For purposes of clarity, the same reference numbers will be used in the drawings to identify similar elements. As used herein, the term module refers to an application specific integrated circuit (ASIC), an electronic circuit, a processor (shared, dedicated, or group) and memory that execute one or more software or firmware programs, a combinational logic circuit, and/or other suitable components that provide the described functionality. As used herein, the phrase at least one of A, B, and C should be construed to mean a logical (A or B or C), using a non-exclusive logical OR. It should be understood that steps within a method may be executed in different order without altering the principles of the present disclosure.

A system 10 for analyzing objects or structures in a bodily fluid sample such as blood is set forth. The system 10 has an image device such as a microscope 12 that is in communication with an analyzer 14. The analyzer 14 is in communication with a model trainer 16. In generally, the microscope 12 is used to obtain images from a sample 20 within a sample holder 18. The analyzer 14 is used for classifying an image using a model generated by the model trainer 16. Although the system is illustrated as three separate components, one or more of the components 12, 14 and 16 may be incorporated into a single unit.

The sample holder 18 may be positioned on a sample positioner 21 such as a table 22. The table 22 is coupled to a position actuator 24. The position actuator 24 may move the table 22 in an XY coordinate. However, the position actuator 24 may also move the table 22 closer to or away from the body of the microscope 12 in XYZ coordinates.

The microscope 12 has a controller 26 that is programmed to control various aspects of the system. The controller 26 is microprocessor-based and is programmed to perform various functions for obtaining images of the sample 20. The sample 20 may be imaged to obtain a plurality of images on video by changing the relative position of the microscope 12 and the sample 20 in a predetermined manner. That is, the position of the microscope 12 may be changed (denoted by the dotted line) or the position of the table 22 may be changed or both.

The microscope 12 also includes a lens 28 that is used for obtaining the image or video. The lens 28 is coupled to the video/image device 30. The video/image device 30 may be a camera such as a digital camera or a digital video camera. A charge couple device may also be used. The video/image device 30 may be a high resolution camera.

The controller 26 may also be used to control the position actuator 24, which may be a plurality of motors. The position actuator 24 is illustrated outside of the microscope 12 and may be coupled to the table 22, the microscope 12, or both. The position actuator 24 may command the table 22 to move in a calculated manner to obtain the proper number of images in predetermined positions. The position determination module 32 controls the sample 20 to be positioned in a predetermined position relative to the microscope 12 and lens 28. The position determination module 32 may be part of or a separate module from the controller 26.

The controller 26 may also control or include a magnification actuator 34. The magnification actuator 34 may control the power of magnification of the microscope 12. Because the system 10 may be used for detecting various objects such as pathogens, the magnification may be required to be changed in order to make such detections of different size objects.

A focus actuator 36 is also in communication with the controller 26. The focus actuator 36 may automatically adjust the focus of the microscope. By adjusting the focus of the microscope, various three dimensional images or videos may be obtained. Focusing the lens 28 accommodate the various depths to allow clear of images to be taken.

A light source controller 38 may be used to control a light source 40. The light source 40 may be one or more different types of light sources having different spectrums with different wavelengths. Visual light or specific wavelengths of light may be generated by the light source 40. For example, ultraviolet light, infrared light or visible light may be generated. Specific wavelengths or combinations of light wavelengths may be used. More than one wavelength of light may be used for detecting specific pathogens. That is, images of a particular sample may be made with more than one wavelength and classified in the analyzer 14.

The analyzer 14 also includes a controller 42. A user interface 44, such as touch screen keyboard or buttons, may be used for initiating functions, actuating various elements and entering various types of data. The analyzer 14 has an image frame determination module 52. The image frame determination module 52 may obtain image frames from a video. Should the video/image device 30 obtain video, various frames (still images) of video may be isolated for image classification. Typically thirty frames per second of video images are generated by a conventional video camera. However, other types of frame rates, including very high frame rates, may be used for classification.

A video processor 54 may receive the video from the video/image device 30 and the image frame determination module 52 may be used to capture various image frames. The captured images may be stored in a memory 56. The memory 56 may be within or outside of the analyzer 14.

A trained image classifier 62 uses a classifier model 60 that has been trained by the model trainer 16 to recognize images. The trained image classifier 62 compares the image to the classifier model 60 to obtain results. The results may be displayed on a display 64. An example of a display 64 for conveying results of the classification are set forth below.

The display 64 and the image classifier 62 may be used to determine various results including the type of objects or pathogens and other data about the objects or pathogen. The shape of the objects or pathogens are used in the determination. A density/severity termination module 66 may use the density or the amount of objects or pathogens within a certain volume to predict severity. A severity indication may be displayed on the display 64. A counter 68 may be used to count the number of detected objects or pathogens in a sample. The count determined by the counter 68 may be used by the density/severity termination module 66 to determine the amount of objects or pathogens within a certain volume.

A confidence generator 70 may generate an indication of confidence in the determination of the presence of objects or pathogens. The confidence generator may generate a confidence value that corresponds to the likelihood of a correct result.

A feedback module 72 may be used as part of the training of the image classifier model 60. That is, feedback module 72, in conjunction with the user interface 44 may allow a user of the analyzer such as a doctor or other health care practitioner to confirm the results or disaffirm the results based on further testing. In this manner, the classifier module 60 may be changed with feedback.

The model trainer 16 has a user interface 80 that is used to provide various data or perform various selections in the generation of the machine learning module 82. An input receiver 84 is used for receiving inputs in the classification process. The inputs may include known specimens that have predetermined numbers and types of pathogens thereon. The learning process will be described in greater detail below.

A network 86 may be used to communicate the classifier model to the analyzer. The network 86 may be a computer network such as the Internet. In this manner, models may be communicated, updated, or new models may be provided from a central location to a number of analyzers 14.

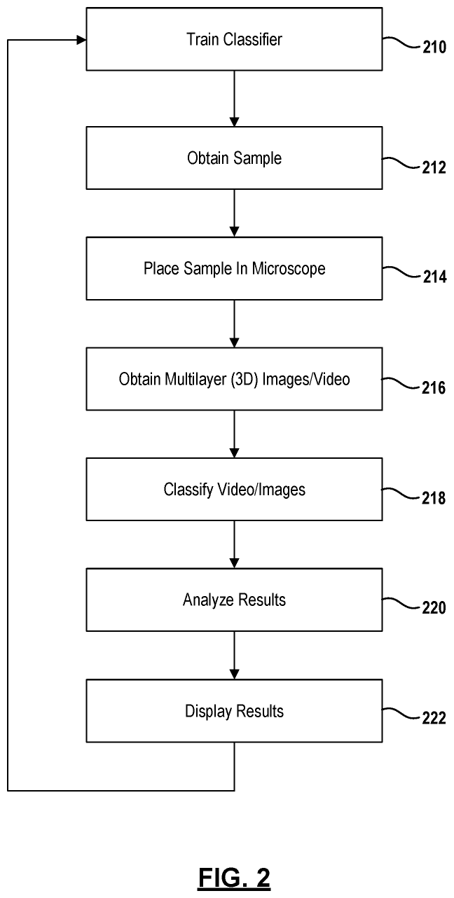

Referring now to FIG. 2, a high level method of operating the system is set forth. In step 210, a classifier is trained. Details of training the classifier is set forth below.

In step 212, a sample of bodily fluid for analysis is obtained as described below. In step 214, the sample is placed near the microscope or other image determination device.

In step 316, a multi-layer or three dimensional video or images are obtained. In step 218, the video or images are classified. The results are analyzed in step 220 and results are displayed in step 222. The results displayed may correspond to the severity or density of the pathogens within the bodily fluid or other indications of the present of a pathogens. Details of the display are also set forth below.

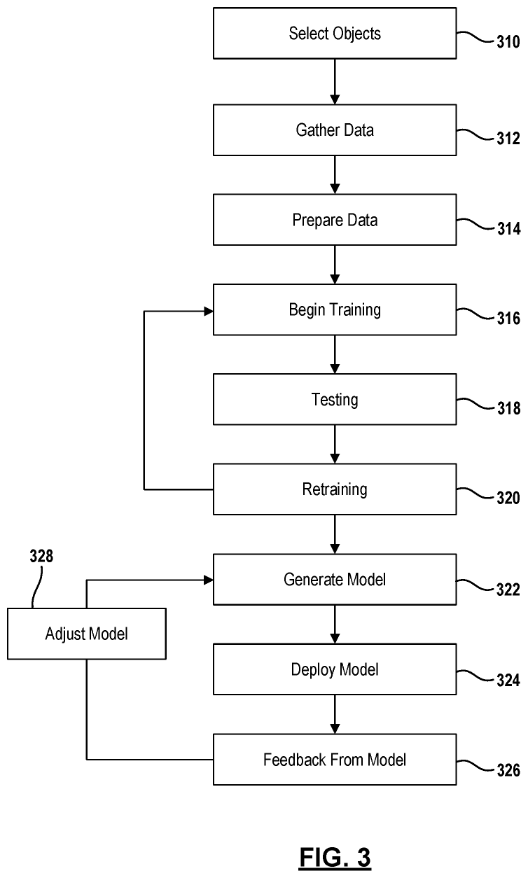

Referring now to FIG. 3, a method for training a classifier is set forth. In step 310, objects are selected for training of the image classifier. Organic structures, objects or bacteria that are visible under a microscope may be used. Ultimately, the selected objects are the objects that are desired to be identified by the analyzing system. In step 312, data is gathered for training of the image classifier. In one constructed example, it was desirable to identify spirochete bacteria and biofilm for the detection of Lyme disease. However, in the training process, symplasts were used for training. The symplasts images were “negative” relative to the model. The symplasts are used in contrast to spirochete and biofilm. Symplasts were to not be identified as either spirochete or biofilms. This is described in more detail below.

In step 314, the data was prepared. The data for the three different categories used in the present example is formatted for proper processing by the model trainer. The syntax such as human readable form is changed to a computer-readable format. Missing, incomplete or corrupted data is also purged from the datasets. The data may be organized for a logical training process. In the present example, videos of spirochete, biofilms and symplasts were used. Still images were obtained from the video. Some of the images were set aside for later use to test the model.

In step 316, the training process is started. A supervised learning approach for entering data may be used. That is, the model trainer may be provided with an image and a description or classification set forth within the image. In this manner, the common characteristics of each different type of pathogen may be determined by the model trainer. Of course, a relative small number to thousands of samples may be provided to a system depending upon the complexity of the pathogens. The factors for the amount of training data required include, but are not limited to, the quality of the training data, the desired sensitivity of the model, the number of categories and the difficulty level of the tasks. In step 318, the model may be tested using some of the data set aside as previously described above. The set aside data is known data and thus the model may be tested to see how it performs. The testing data is not used in the training of the model. Typically, a considerable amount of testing data may be set aside for testing purposes. For example, about 20% to about 30% of data may be reserved for testing rather than training. During testing, when the model is not performing adequately, the system may be retrained in the retraining step 320. Retraining takes place by performing the training step 316 and the testing step 318 repeatedly until adequate results are provided. It is desired to prevent “overfitting” of the data. That is, if too much training is provided, a danger of false negatives may also be obtained.

After each training iteration, the testing step 318 is performed and the results are compared to the previous training iterations.

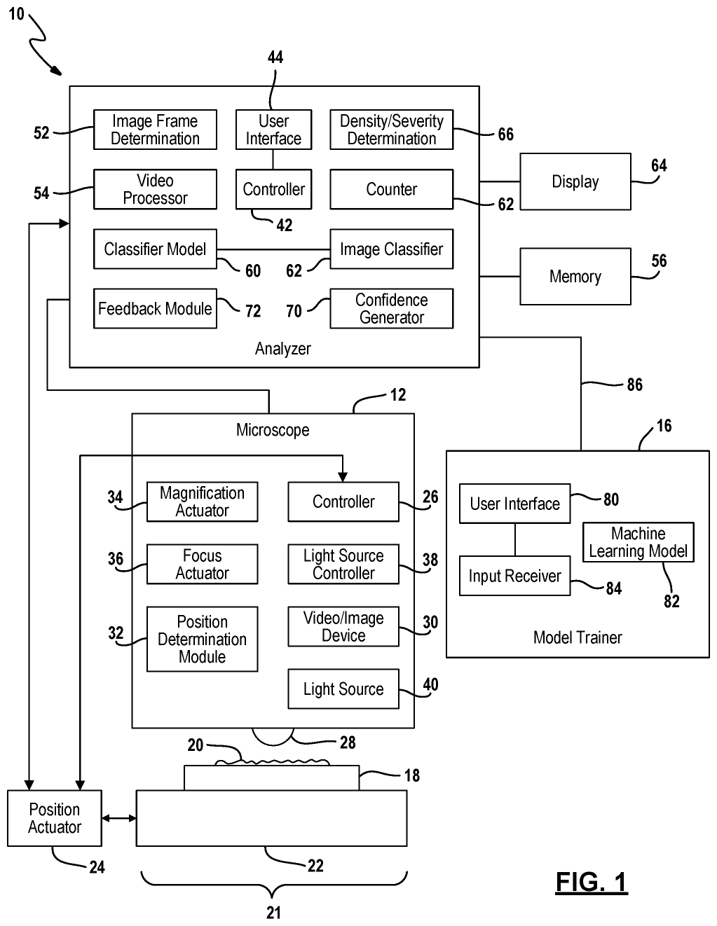

In step 322, the model is ultimately generated based on the training and the retraining steps provided above. In step 324, the model may be deployed. That is, once the classifier model is trained to the desired state of performance, the model may be communicated to the analyzer and used in real world classification. In a health care setting, the model trainer 16 may be located at one location and the image classifier may be located at another location such as within a doctor's office. Updated classifier data may be communicated through the network 86 illustrated in FIG. 1.

In step 326, once the model has been provided in a clinical type setting, feedback may be obtained. Feedback may be obtained for the model based upon use and further testing using a different type of system. In step 326, feedback may be provided based upon additional testing. The feedback may be used to adjust the model in step 328. That is, the adjustment of the model may be performed and taken into consideration in step 322.

The model is adapted and benefits through multiple training using data as well as benefitting from feedback. The feedback may also be communicated back to the model trainer through the network 86 to make the model for future applications better. Feedback may be provided to the model trainer through images and data corresponding to the images such as positive or negative results. This allows the image classifier to train and retrain itself in real time using the feedback and better informed calculations. Continuous refinements allow better results to be obtained in the future.

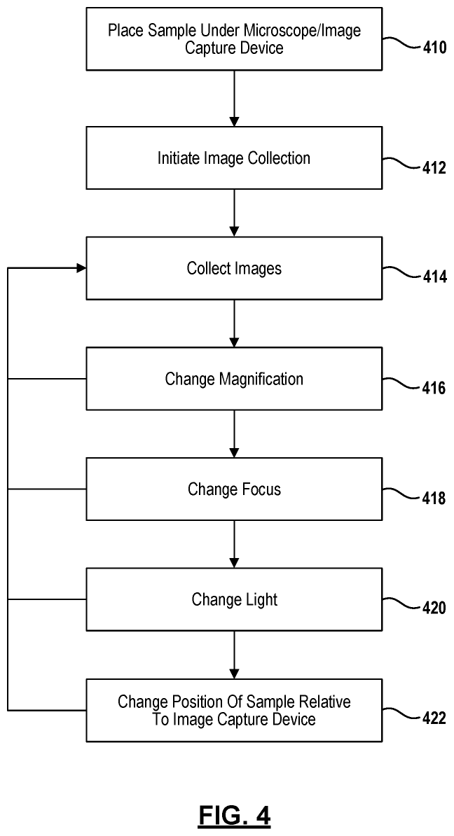

Referring now to FIG. 4, in step 410, a sample is placed within view of the microscope or image capture device. As mentioned above, the microscope 12 or image capture device may have a table 22 that moves in various planar directions relative to the lens 28. This allows a density of the pathogens to be determined for a predetermined volume. In step 412, image collection is initiated. The initiation of the image collection may be performed at the analyzer 14 described above. The user interface 44 of the analyzer may be used to initiate the process and select the pathogen or pathogens desired to be protected. The image collection may be initiated by obtaining a video of a predetermined sample and the collecting images in step 414 from the video. Of course, still images may be obtained directly rather than from a video.

In step 416, the magnification of the microscope may be varied depending upon the desired size of the pathogen. If more than one pathogen is selected, the magnification may change during the process. Multiple images may be collected in step 414 by varying the various perimeters of the microscope or image collection device. For example, in step 416, the magnification may be changed multiple times during obtaining of images to detect various sizes of pathogens. In step 418, the focus may be changed to focus at various levels or various positions within a sample. In step 420, the lighting may be changed. Multiple types of light sources, as described above, may be used. Images may be taken from a sample using more than one wavelength of light. That is, an image may be taken with one wavelength and a second image with the same settings at the same location may be taken with a different wavelength. The proper wavelength may change depending on the particular pathogen.

In step 422, the position of the sample relative to the image capture device is changed. The position actuator 24 described in FIG. 1 may be used to change the position of the table 22 relative to the lens 28 of the microscope or imaging device. Images may be taken from various parts of the sample including higher or lower depths and various XY positions. This allows a three dimensional set of images to be obtained. Again, the images may be obtained from a video and the video taken while the position actuator 24 moves the table 22.

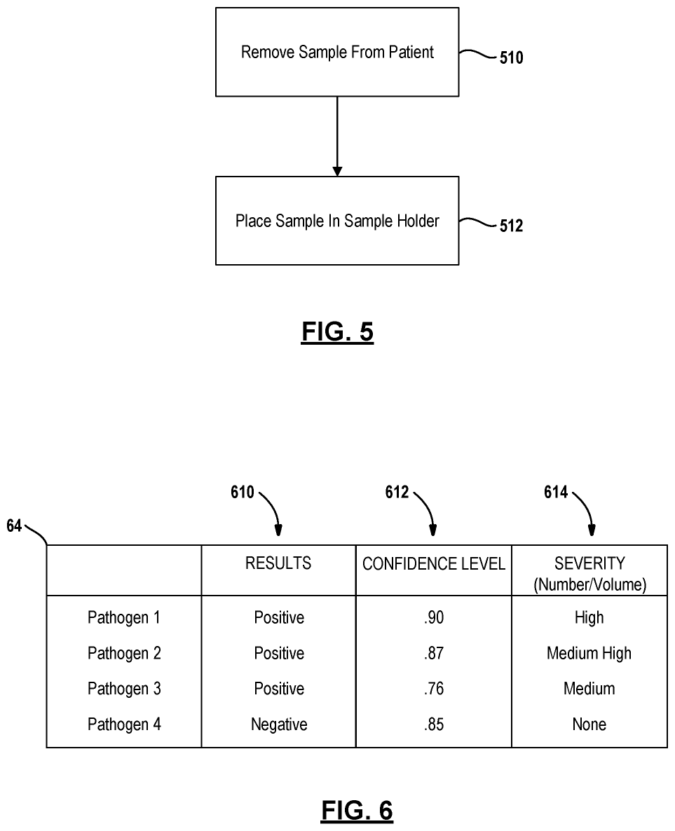

Referring now to FIG. 5, a method for obtaining a sample is set forth. In step 510, a sample is removed from the patient. The sample may be removed by a needle or withdrawing a blood from the patient. If other bodily fluids are used, other methods for obtaining the bodily fluids may be used. In step 512, the sample is placed in a sample holder. The sample holder may, for example, be a slide or well type device. For certain pathogens, time may be critical due to breaking down of the objects and thus the analyzing may be performed within minutes of obtaining the sample. The analyzer may be located in a lab or doctor's office so that easy access to a free sample may be provided.

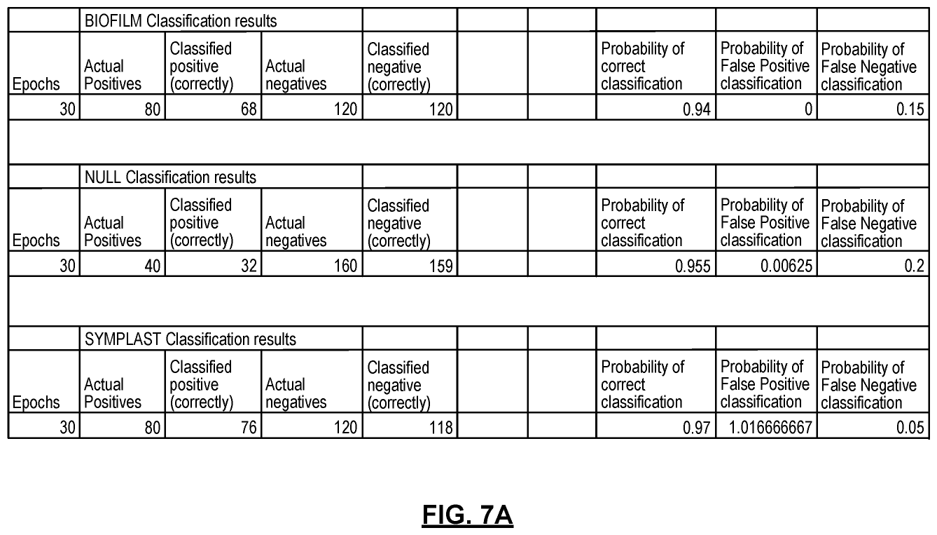

Referring now to FIG. 6, the screen display 64 for providing a sample is set forth. In this example, the screen display 64 is used for displaying results for four different types of pathogens. One or more than one type of pathogen may be tested. In the simplest sense, a positive or negative result may be provided in column 610. In this example, three positive results and one negative result corresponding to the respective pathogens 1-4. A confidence level column 612 may also be provided. The confidence levels set forth in the display 64 correspond to the confidence for which the positive or negative results are then provided. In this example, pathogen 1 has a confidence level of 0.9, pathogen 2 0.87, pathogen 3 7.6 and pathogen 4 4.85. The confidence level corresponds to the level that the classifier performs. The confidence level may correspond to the amount of training of the image classifier. A severity may also be generated. The severity may correspond to a number or the volume. In this example, the severity corresponds to high, for the first pathogen, medium high, for the second pathogen, medium, for the third pathogen, and none for the fourth pathogen. Of course, the density which is the number of pathogens detected in a volume may also be generated and displayed numerically. The severity is set forth in column 614.

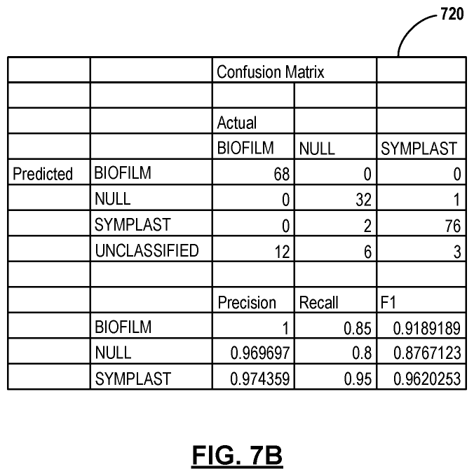

Referring now to FIG. 7A, one example performed for training a model used ResNet on microscope images in three different classes. In this example, class 1 was a biofilm, class 2 were symplasts and class 3 was a null class. Biofilm is a residue. The symplasts are a crystallized organic structure. The null class was comprised of various microorganisms that are not biofilms or symplast. 400 images of biofilm, 400 images of the null class and 400 images of symplasts were used. 133 images of each were used for validation. Using the ResNet based network, a 90% accuracy rate was achieved for biofilm, 80 actual positives, 68 classified positives (correctly), 120 actual negatives and 120 classified negative (correctly) were achieved. Thus, the probability of a correct classification was 0.94, the probability of false/positive classification was 0 and the probability of false/negative classification was 0.15. For the null classification and the symplasts classification results, see FIG. 7A.

Referring now to FIG. 7B, a confusion matrix 720 shows that the model predicted most of the images correctly according to the designated classes. Classifications of unclassified is set forth when the confidence is below a threshold of 80%.

Various hyperparameters were used in training the model. 30 epochs were used. 3,540,720 image shapes were used. A learning rate of 0.001 was also used. A mini-batch size of 25, the number of classes was 3, the number of layers was 152, the number of numeral training samples was 1200 and the use pretrained model was 1. Of course, different types of objects, structures and pathogens will require different amounts of training.

While the present example is directed to blood, other bodily fluids may be sampled with the disclosed system and in accordance with the disclosed system and method.

Those skilled in the art can now appreciate from the foregoing description that the broad teachings of the disclosure can be implemented in a variety of forms. Therefore, while this disclosure includes particular examples, the true scope of the disclosure should not be so limited since other modifications will become apparent to the skilled practitioner upon a study of the drawings, the specification and the following claims.

1. A system comprising:
a sample holder holding a bodily fluid sample;
an image capture device generating a plurality of images of the sample;
a sample positioner positioning the sample holder;
image capture device generating a plurality of images of the sample at a plurality of positions;
a trained image classifier classifying the plurality of images to identify a type of objects in the bodily fluid sample
an analyzer, that in response to classifying, displays an indicator on a display for indicating the type of objects is present within the bodily fluid sample.

2. The system of claim 1 wherein the bodily fluid comprises blood and the type of objects comprise pathogens.

3. The system of claim 1 further comprising a controller coupled to the image capture device, said controller controlling the sample positioner into the plurality of positions.

4. The system of claim 1 wherein the controller controls a focus of the image capture device, controls magnification of the image capture device and wherein the image capture device generates images at different focuses, different magnifications and at different positions.

5. The system of claim 1 wherein the sample positioner controls the position so that the image capture device generate images within a three dimensional volume of the bodily fluid sample.

6. The system of claim 1 wherein the image capture device generates images from a video of the sample.

7. The system of claim 1 wherein the indicator corresponding to object density within the bodily fluid sample.

8. The system of claim 1 wherein the trained image classifier generates a score for each of the plurality of images and an overall score using each score for each of the plurality of images.

9. The system of claim 1 wherein the trained image classifier determines a confidence level of classifying and wherein the indicator comprises the confidence level.

10. The system of claim 1 wherein the indicator corresponds to a severity based on density of the objects.

11. A method comprising:
controlling a position of an image capture device relative to a bodily fluid sample;
generating a plurality of images from the bodily fluid sample at a plurality of different positions;
classifying the plurality of images at a trained image classifier to identify a type of object in the bodily fluid sample; and
in response to classifying, displaying an indicator on a display associated with an analyzer for indicating the type of object is present within the bodily fluid sample.

12. The method of claim 11 wherein classifying the plurality of images at the trained image classifier to identify objects therein and wherein classifying the plurality of images at the trained image classifier to identify a pathogen therein.

13. The method of claim 11 further comprising controlling a focus of the image capture device, controlling magnification of the image capture device and wherein generating images comprises generating images at different focuses, different magnifications and at different positions.

14. The method of claim 11 wherein controlling the position comprises controlling the position to generate images in multiple layers of the bodily fluid sample.

15. The method of claim 11 wherein controlling the position comprises controlling the position to generated images within a three dimensional volume of the bodily fluid sample.

16. The method of claim 11 further comprising generating a video of the sample and wherein generating the plurality of images comprises generating the plurality of images from the video of the sample.

17. The method of claim 11 wherein generating the plurality of images comprises generating a video of the sample and obtaining still images from the video at different focuses and magnifications.

18. The method of claim 11 wherein displaying the indicator comprises displaying the indicator corresponding to object density within the bodily fluid sample.

19. The method of claim 11 wherein further comprising determining an object density and wherein generating the indicator comprises generating the indicator corresponding to the object density within the bodily fluid sample.

20. The method of claim 11 wherein classifying the plurality of images comprises generating a score for each of the plurality images and an overall score using each score for each of the plurality of images.

21. The method of claim 11 further comprising determining a confidence level of classifying and wherein displaying the indicator comprises displaying the confidence level.

22. The method of claim 11 further comprising determining a density of objects within the bodily fluid sample and wherein displaying the indicator comprises displaying the indicator indicative of severity based on the density.
